(19) United States
(12) Plant Patent Application Publication
(10) Pub. No.: US 2003/0226183 P1
(43) Pub. Date: Dec. 4, 2003

(54) Gaura lindheimeri plant named 'COLSO'

(76) Inventor: Howard Bentley, Victoria (AU)

Correspondence Address: CHRISTIE, PARKER & HALE, LLP, 350 WEST COLORADO BOULEVARD, SUITE 500, PASADENA, CA, 91105, US

(21) Appl. No.: 10/160,952

(22) Filed: May 30, 2002

(51) Int. Cl. A01H 5/00

(52) U.S. Cl. PLT/226

(57) ABSTRACT

Described is a new Gaura Lindheimeri plant having variegated foliage with a central main color of green and an outer edge leaf color of yellow-white.

### BACKGROUND OF THE INVENTION

The present invention relates to a new Gaura Lindheimeri plant having the varietal denomination ‘COLSO.’ ‘COLSO’ was discovered as a seedling variant of the variety Gaura Lindheimeri ‘SISKIYOU PINK’ in a controlled planting.

### SUMMARY OF THE INVENTION

The new variety of a Gaura Lindheimeri plant is an evergreen perennial shrub which has a medium sparse, spreading, growing habit distinguished by its variegated foliage. Asexual reproduction of the new variety by vegetative cuttings in Wonga Park, Victoria, Australia has demonstrated that the distinguishing characteristics are transferred through succeeding generations.

### COMPARISON WITH PARENT

The new variety differs from its parent, ‘SISKIYOU PINK’, in having variegated foliage.

### BRIEF DESCRIPTION OF ILLUSTRATIONS

The accompanying illustrations depict specimen plants of the new variety in color as true as possible in color photographic illustrations of this nature.

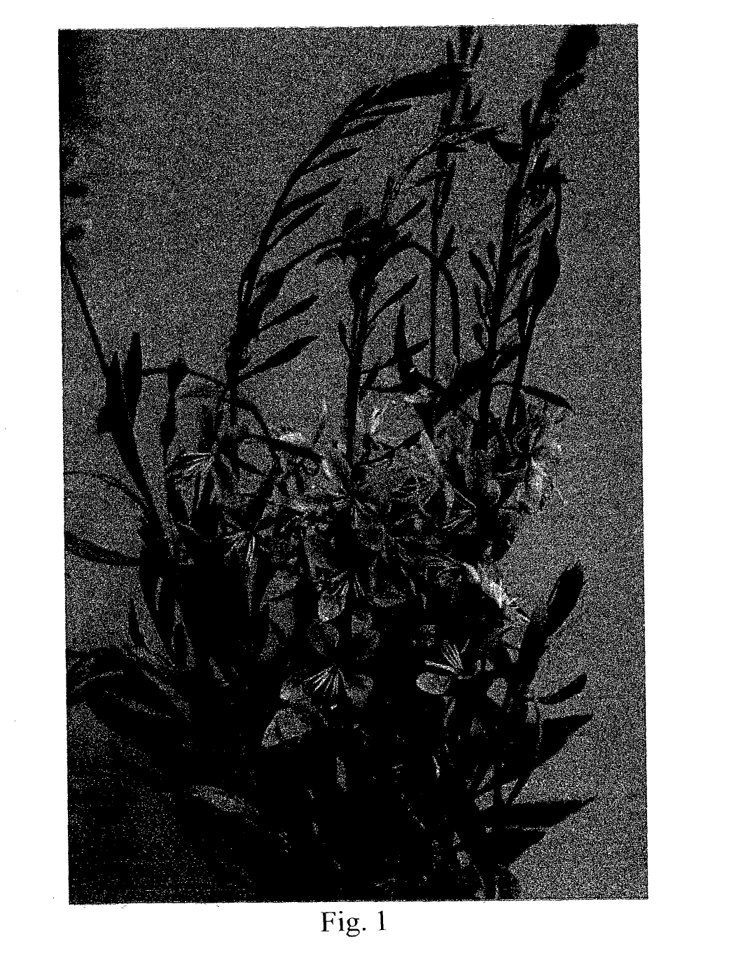

FIG. 1 shows the plant of the new variety; and

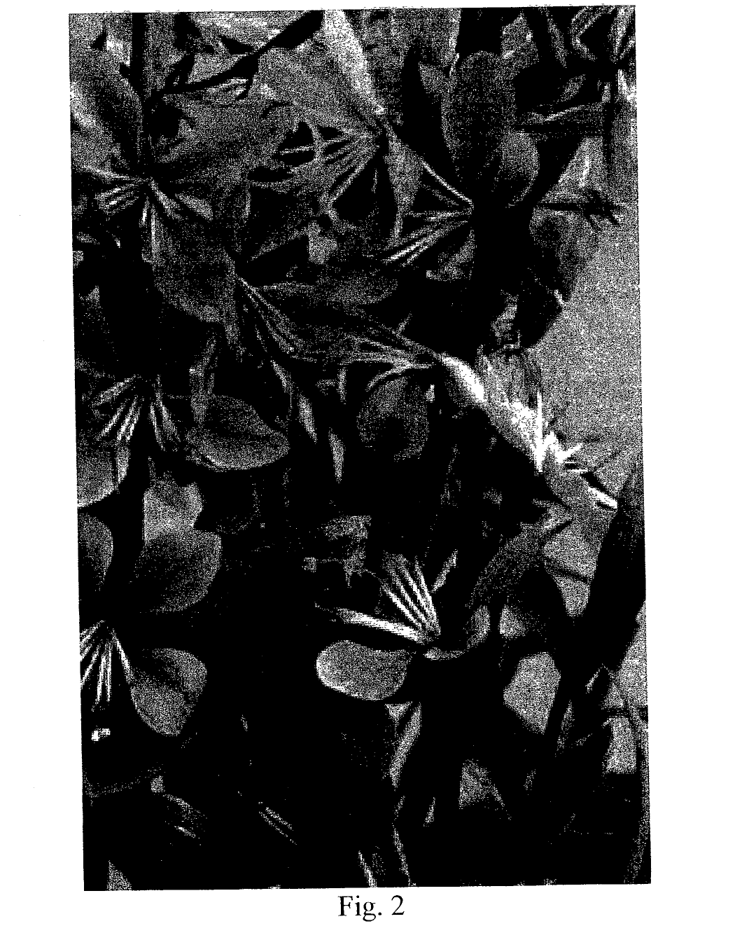

FIG. 2 shows a close up of the flower and variegated foliage of the new variety.

### DESCRIPTION OF THE NEW VARIETY

The following is a description of the new variety taken from plants grown out-of-doors in Wonga Park, Victoria, Australia during the month of April. Color designations used herein are from the 1995 Royal Horticultural Society Color Chart (“RHSC”).

### PLANT

The plant grows in a spreading habit with medium sparse, vigorous growth, attaining medium height.

### FOLIAGE

Leaflet:

Size.—Medium.

Quantity.—Medium to sparse.

Color:

Leaflet center.—Near 137C.

Leaflet outer edge.—Near 158A.

Shape: Leaves are linear to lanceolate.

Texture:

Upper side.—Smooth with fine pubsescents.

Underside.—Smooth with fine pubsescents.

Veination: Reticulate.

Edge: Slight undulate margins observed.

### WOOD

New shoots:

Color.—137B and 137C.

Bark.—Smooth with fine pubsescents.

Old wood:

Color.—199A.

Bark.—Smooth with fine pubsescents.

Winter hardiness: Winter hardy to above −5° C.

Resistance to disease: Good, observed to be generally disease resistant.

### REPRODUCTIVE ORGANS

Arrangement: Stamens, filaments and anthers are regularly arranged at the base, but bunched in a pendular formation.

Filaments: White to pale pink.

Anthers: Crimson to brown.

Pollen: Yellow.

Styles: Thin, singular, long length.

Stigma color: Pale yellow.

Hips:

Shape.—Oval to linear.

Size.—Small with no seed protrusion.

Flower: Four petals, color near 65A with petal veins near 66C.

I claim:

1. A new Gaura Lindheimeri plant of the variety substantially as shown and described.
